(19) United States
(12) Patent Application Publication
(10) Pub. No.: US 2017/0079531 A1
(43) Pub. Date: Mar. 23, 2017

(54) Flavored Sleeve Assembly

(71) Applicant: Lori A. Scarborough, Woodbury, TN (US)

(72) Inventor: Lori A. Scarborough, Woodbury, TN (US)

(21) Appl. No.: 14/858,028

(22) Filed: Sep. 18, 2015

(51) Int. Cl. A61B 5/01; G01K 1/08; A23G 3/36

(52) U.S. Cl. CPC A61B 5/01; A23G 3/36; G01K 1/083

(57) ABSTRACT

A flavored sleeve assembly for imparting a flavor to an oral thermometer includes a sleeve that may be slid onto a temperature probe when the temperature probe is inserted into a mouth. A flavor layer is coupled to the sleeve. The flavor layer imparts a flavor in the mouth thereby enhancing an appeal of having the sleeve being positioned within the mouth.

### BACKGROUND OF THE DISCLOSURE

### Field of the Disclosure

The disclosure relates to sleeve devices and more particularly pertains to a new sleeve device for imparting a flavor to an oral thermometer.

### SUMMARY OF THE DISCLOSURE

An embodiment of the disclosure meets the needs presented above by generally comprising a sleeve that may be slid onto a temperature probe when the temperature probe is inserted into a mouth. A flavor layer is coupled to the sleeve. The flavor layer imparts a flavor in the mouth thereby enhancing an appeal of having the sleeve being positioned within the mouth.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective view of a flavored sleeve assembly according to an embodiment of the disclosure.

FIG. 2 is a back view of an embodiment of the disclosure.

FIG. 3 is a cross sectional view taken along line 3-3 of FIG. 2 of an embodiment of the disclosure.

FIG. 4 is a perspective in-use view of an embodiment of the disclosure.

FIG. 5 is an exploded perspective view of an embodiment of the disclosure.

### DESCRIPTION OF THE PREFERRED EMBODIMENT

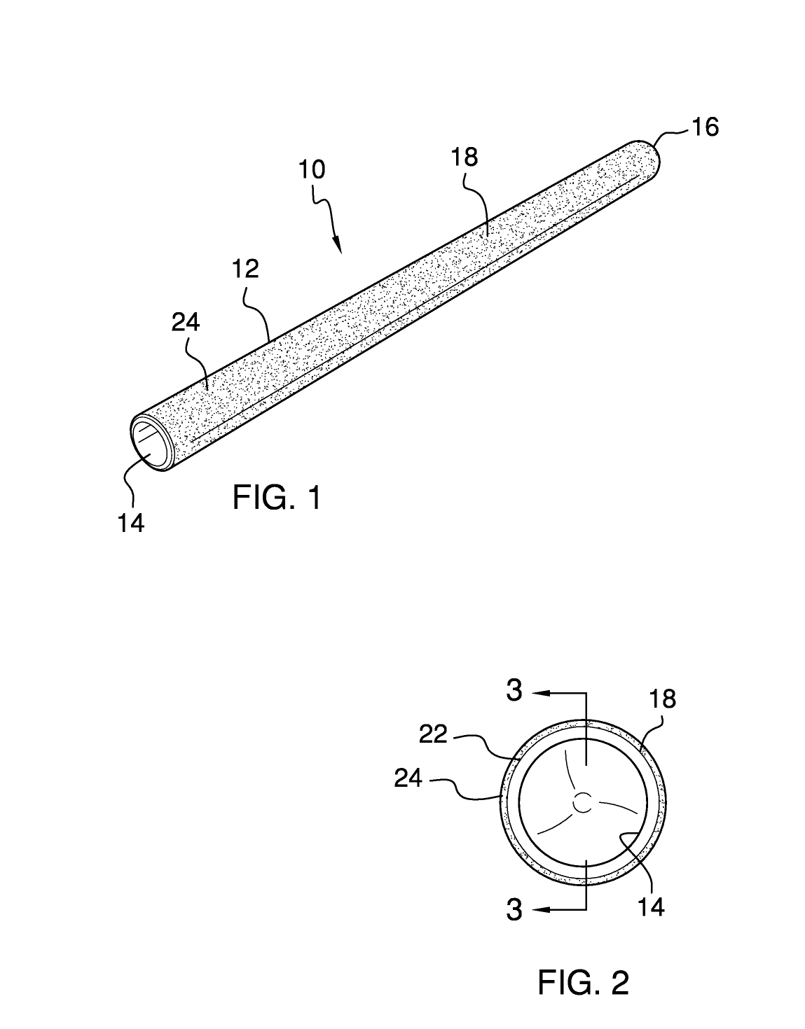

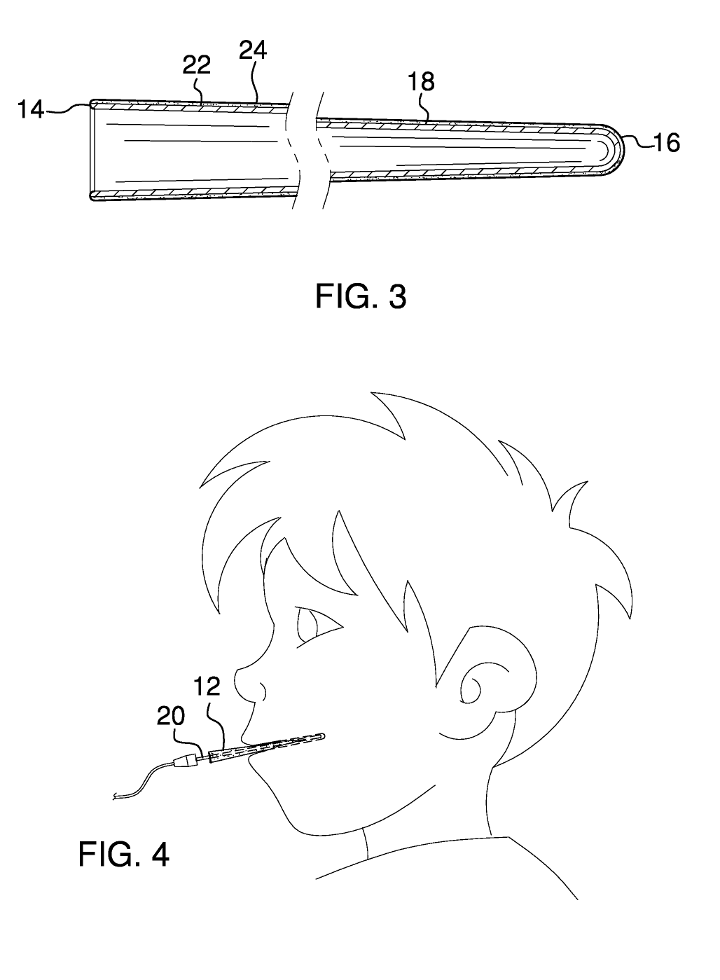

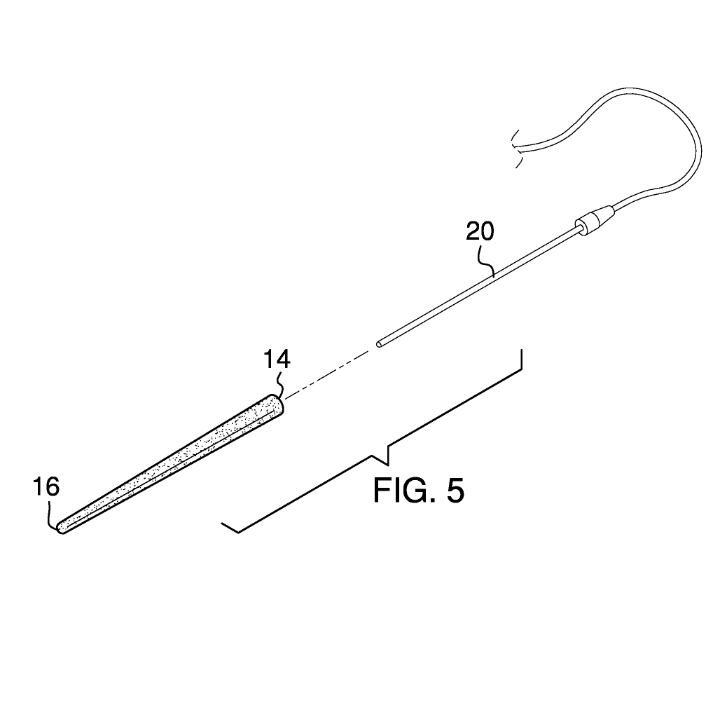

With reference now to the drawings, and in particular to FIGS. 1 through 5 thereof, a new sleeve device embodying the principles and concepts of an embodiment of the disclosure and generally designated by the reference numeral 10 will be described.

As best illustrated in FIGS. 1 through 5, the flavored sleeve assembly 10 generally comprises a sleeve 12 that has a first end 14, a second end 16 and an outer wall 18 extending between the first end 14 and the second end 16. The outer wall 18 is curved such that the sleeve 12 has a cylindrical shape. The first end 14 is open and the sleeve 12 may be slid onto a temperature probe 20 when the temperature probe 20 is inserted into a mouth. The temperature probe 20 may comprise an electronic temperature probe utilized in the medical field. The outer wall 18 has an outer surface 22 and the outer wall 18 tapers inwardly between the first end 14 and the second end 16.

A flavor layer 24 is coupled to the sleeve 12. The flavor layer 24 is infused with a chemical flavor. Thus, the flavor layer 24 imparts a flavor in the mouth thereby enhancing an appeal of having the sleeve 12 being positioned within the mouth. The flavor layer 24 is positioned on the outer surface 22 and the flavor layer 24 completely covers the outer surface 22. The flavor layer 24 may have a grape flavor, a cherry flavor, a bubblegum flavor, a mint flavor or other flavor. Additionally, the flavor layer 24 may be color coded to indicate the flavor of the flavor layer 24.

In use, the sleeve 12 is positioned on the temperature probe 20. The temperature probe 20 is inserted into the mouth and the flavor layer 24 imparts the flavor into the mouth. The flavor layer 24 serves to reduce anxiety and resistance to having the temperature probe 20 being inserted into the mouth. The sleeve 12 is removed from the temperature probe 20 and the sleeve 12 is discarded when temperature of the mouth is recorded.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A flavored sleeve assembly being configured to be removably coupled to a thermometer, said assembly comprising:
a sleeve being configured to be slid onto a temperature probe when the temperature probe is inserted into a mouth; and
a flavor layer being coupled to said sleeve wherein said flavor layer is configured to impart a flavor in the mouth thereby enhancing an appeal of having said sleeve being positioned within the mouth.

2. The assembly according to claim 1, wherein said sleeve has a first end, a second end and an outer wall extending between said first end and said second end, said outer wall being curved such that said sleeve has a cylindrical shape, said first end being open.

3. The assembly according to claim 2, wherein said outer wall has an outer surface, said outer wall tapering inwardly between said first end and said second end.

4. The assembly according to claim 1, wherein:
said sleeve has an outer surface; and
said flavor layer being positioned on said outer surface, said flavor layer completely covering said outer surface.

5. A flavored sleeve assembly being configured to be removably coupled to a thermometer, said assembly comprising:
a sleeve having a first end, a second end and an outer wall extending between said first end and said second end, said outer wall being curved such that said sleeve has a cylindrical shape, said first end being open wherein said sleeve is configured to be slid onto a temperature probe when the temperature probe is inserted into a mouth, said outer wall having an outer surface, said outer wall tapering inwardly between said first end and said second end; and
a flavor layer being coupled to said sleeve wherein said flavor layer is configured to impart a flavor in the mouth thereby enhancing an appeal of having said sleeve being positioned within the mouth, said flavor layer being positioned on said outer surface, said flavor layer completely covering said outer surface.
